(19) United States
(12) Patent Application Publication
(10) Pub. No.: US 2008/0306506 A1
(43) Pub. Date: Dec. 11, 2008

(54) SELF-INFLATING AND DEFLATING INTRAGASTRIC BALLOON IMPLANT DEVICE

(76) Inventor: Dennis A. Leatherman, Palm Springs, CA (US)

Correspondence Address: DENNISON, SCHULTZ & MACDONALD, 1727 KING STREET, SUITE 105, ALEXANDRIA, VA, 22314, US

(21) Appl. No.: 11/760,905

(22) Filed: Jun. 11, 2007

(51) Int. Cl. A61M 29/02

(52) U.S. Cl. 606/192

(57) ABSTRACT

A variable-sized, self-inflating and self-deflating intragastric balloon device, includes a hiocompatible polymer membrane formed into a balloon having inner and outer surfaces preferably coated with a wax, the balloon defining an interior lumen having an internal capsule freely floating within the lumen, the internal capsule having first and second chambers with a communicating one way valve therebetween. The first chamber is formed from a biocompatible polymer preferably coated with wax forming a gas and liquid tight housing to store a liquid solution therein, and the second chamber is composed of material capable of self dissolving or degrading in the presence of the liquid solution. Application of external pressure will transfer liquid from the first chamber through the valve into the second chamber where it will degrade the chamber and react with a solid reagent disposed within the lumen external to the capsule, forming a gas which inflates the balloon. A manufactured weakness within the membrane wall reacts chemically and/or physically causing a deflation of the balloon.

### BACKGROUND OF THE INVENTION

1. Field of the Invention

The invention relates to the field of intragastric balloon implants used to assist in loss of weight, and in particular, balloon implants which are inflated after placement in the body.

2. Description of Related Art

Intragastric balloons that are placed in the overweight person yield measurable weight loss. The occupation of a specific volume of space within the stomach by the intragastric balloon diminishes the appetite and provides a feeling of satiety resulting in weight loss. This loss of weight is notably enhanced with the modification of eating habits along with alterations to the person's life style.

The original intragastric balloons were externally inflated and deflated, and have a number of undesirable effects that have plagued researchers and surgeons for decades.

Safety is the most important factor facing the weight loss community that is considering the implantation of an intragastric balloon. Safety risks are inherently a part of the procedure itself, including the anesthesia associated with the procedure, the risk to the patient's overall health and the potential complications and side effects including premature or unexpected deflation of the device resulting in its passage into the small intestine, causing intestinal blockage requiring invasive surgery. On occasion, these intestinal blockages have resulted in death.

While the procedure for implanting the balloons is not a surgical operation, it nevertheless is exacting and complex. Unlike outpatient surgical procedures that require a single visit to the operating room or special procedures room and afterwards a stay in the recovery room, the placement of an intragastric balloon that is externally inflated and/or deflated or otherwise externally modulated requires at least two visits, one for insertion and one for extraction, to the hospital operating room or special procedure room and post procedure recovery room. This results in added costs, potential medical and surgical complications and inconvenience to the individual.

Moreover, the procedure yields a short term weight loss due to its temporary residency in the individual's stomach. The temporary weight loss benefit when compared to the risk factors involved with the procedure makes it difficult to justify the multiple procedures and the associated risk of multiple interventions.

After the placement of the intragastric balloon, there are varied patient discomforts associated with the mechanical properties of the device. These side effects include, but are not limited to, sensations of nausea and vomiting, gastric cramping and ulceration of the stomach mucosa as well as intestinal blockage due to unplanned mechanical deflation. These side effects are largely due to a “one-size-fits-all” approach to these externally modulated devices. Options in sizing is a benefit that would alleviate at least some of the patient discomfort that is experienced under current inflexible device designs.

Newer intragastric balloons are designed to be self inflating and self deflating. A self inflating balloon is disclosed in U.S. Pat. No. 5,129,915, which is directed to an intragastric balloon that is swallowed and inflates according to temperatures within the body. The reactive agents are acid, water and a carbonate or bicarbonate. These reagents are separated from the water by coatings of chocolate, cocoa paste, or cocoa butter which break down and mix together over time within the body. The alternative reagents are citric acid and bicarbonate coated with animal or vegetable fat that breaks down under body temperature permitting the chemical reaction as above. A final proposal is solid acid and bicarbonate that are maintained separate by a pouch of synthetic material which is broken immediately before swallowing the capsule. Breaking the capsule permits the mixture of the reagents, causing the gas to initiate the inflation of the balloon immediately; this increases the likelihood of esophageal blockage. While heat is an effective way to induce the breakdown of coatings selected, allowing the mixing of the reagents to inflate the balloon, it is generally unreliable in its timing and therefore subjects the patient to unnecessary risk.

U.S. Pat. No. 6,981,980 is directed to a self inflating intragastric balloon in which the activating solution is injected by a syringe through a septum, into a capsule that deteriorates or breaks down permitting the mixture of the chemical agents, forming a gas to inflate the balloon. It is delivered in a kit form to the medical professional.

The self-inflating and self-deflating device presents new safety concerns.

In the development of these self inflating-deflating devices, there are two issues that plague researchers. The most significant is the untimely inflation of the balloon in the esophagus prior to entrance into the stomach. As in the case of the untimely deflation of the externally modulated intraqastric balloon that has resulted in intestinal blockage and death, the untimely inflation of the self inflating-deflating intragastric balloon in the esophagus presents equally serious health concerns.

A second issue is the potential for delayed inflation of the balloon resulting in passage of the balloon into the intestinal tract and inflating therein causing bowel blockage and other complications. In the event that the un-inflated balloon passes through the alimentary tract and is evacuated from the body, all of the anticipated benefits of the balloon experience are void in this attempt at weight loss. This reference to untimely inflation is particularly pronounced in the limited control offered by heat-only activated devices.

The implantation procedure for self-inflating balloons is also exacting and complex. Some mechanical devices that self inflate-self deflate present protuberances or hard configured projections housed within the balloon structure and which are made an integral part of the primary device. These injectable insertion points that are hardened and protrude into the lumen of the balloon are more likely to cause a blockage or perforation of the alimentary tract than a device that has none. This possibility of blockage or perforation can lead to subsequent surgery and anesthesia in treating the resulting complications.

The procedure yields a short term weight loss benefit compared to the possible complications of premature inflation causing pressure on the heart, lungs and other related soft tissues surrounding the esophagus. It can further result in similar tissue obstructive problems in a delayed inflation. This temporary weight loss benefit makes it difficult to justify the initial and furthermore repeated procedures and the associated risks of all of these interventions.

Moreover, to date, the engineering and other requirements for manufacturing the self inflation-deflation devices have been complex, difficult and costly to accomplish.

What is needed is a simple, easy to manufacture, safe and affordable self inflating-deflating device, sizable to the individual that solves or significantly diminishes all of the above problems, including emergency evacuation of the device in the event of premature inflation in the esophagus and mechanical and chemical and physical assurance to prevent delayed inflation in the intestinal tract. It is important that delayed inflation and passage of the inflated device into the intestinal tract be prevented or remedied, if it has occurred. It is also important to avoid early inflation in the esophagus.

### SUMMARY OF THE INVENTION

It is therefore an object of the invention to provide a simple and safe intragastric balloon that will self inflate within the stomach, utilize non-toxic agents and reagents, produce non-toxic by-products and gases, bond the intragastric balloons together in such collective volume to yield significant occupancy within the stomach to affect weight loss and promote the avoidance of premature evacuation from the stomach and provide for emergency evacuation of the balloon from the esophagus if ever needed.

It is a further object of the invention to provide a balloon which is simple to engineer, produce, ingest, and evacuate.

To achieve these and other objects, the invention is directed to a variable-sized, self-inflating and self deflating intragastric balloon device, comprising:

a biocompatible polymer membrane formed into a balloon having inner and outer surfaces coated with a wax, the balloon defining an interior lumen;

an internal capsule freely floating within the lumen and comprising first and second chambers with a communicating one way valve between the chambers, with the first chamber comprising a biocompatible polymer coated with wax forming a gas and liquid tight housing to store a liquid solution therein, and the second chamber being composed of material capable of self dissolving or degrading in the presence of a liquid solution, wherein application of external pressure will transfer liquid from the first chamber through the valve into the second chamber;

a solid reagent disposed within the interior portion external to the capsule, the solid reagent reacting with the liquid solution forming a gas which inflates the balloon; and

a manufactured weakness within the membrane wall or a material design and use of a specific material that will permit chemical reaction with the weakness or material design, or the specific material causing a predetermined deflation.

According to the invention, within a predetermined period of time, the membrane or the material design of the balloon will be degraded by chemical interaction, mechanical pressures or external forces on the balloon to cause deflation. This may be accomplished by one or more of the following means:

an engineered weakness or perforation or a manufactured hole and plug combination in the balloon membrane wall that is both mechanically and chemically activated;

a selection of specific bonding agents, glues, and processes or a combination thereof in the sealing process of the device that will promote a timed partial weakening or degradation of the membrane permitting timed deflation; or

the balloon material may be selected such that application of external forces such as sonic waves, heat or any other viable focused energy causes deflation to occur in a safe manner.

The balloon device will be loaded into a dissolvable capsule, or otherwise coated and bound before swallowing.

The intragastric balloon is made of a biocompatible envelope, such as polyethylene, PVC, PVCD, PET, Teflon, polypropylene, silicone or any other appropriate material. The membrane is liquid and gas impermeable, which is effected by dipping the membrane into a wax, preferably ultra-filtered beeswax, that will meld thereto. This gas/liquid tight balloon is capable of insuring against contamination of the agents and/or reagents by liquids and humidity. Within the balloon is a lumen that is dry and will house the required chemical agents that will be used to create the gas for inflation of the balloon. This inflation will be accomplished chemically and automatically without any external devices, syringes, requirements or attachments. The invention also provides for separation of the agents depending upon manufacturer's placement of the solid chemicals within the balloon. No design modifications are required to cause this separation of the agents until the liquid is released from the second chamber.

The subsequent chemical reaction will be effected by controlled temperature and physical conditions. The invention calls for placement of a dual chamber liquid-filled capsule within the lumen of the balloon. The capsule is detached and free floating, and will include two chambers joined by a self-sealing unidirectional valve. The first chamber is made of a biocompatible material, such as polyethylene, PVC, PVCD, PET, Teflon, polypropylene, silicone or any other appropriate material that is coated with a wax or other substance that is biocompatible and that will prevent permeability of the membrane to both gases and liquids. The second chamber is a capsule that is dissolvable by any non-toxic liquid substance. The transfer of the liquid substance from the first to the second chamber is accomplished by mechanical force, and the transferred liquid solution will cause the automatic dissolving of the membrane of the capsule after a predetermined time. The capsule will be made from any appropriate, biocompatible material such as a gelatin which dissolves upon exposure to the liquid in the capsule. Upon degradation of the membrane, the liquid substance will automatically mix with reagent housed within the lumen of the balloon, causing chemical conversion of the agents into a gas such as carbon dioxide that will automatically inflate the balloon.

Because humidity and moisture content are at issue, the membrane forming the wall of the intragastric balloon and the membrane forming the wall of the first chamber must be liquid and gas tight.

Degradation of the balloon will cause the gradual release of its non-toxic gases and possibly the remaining material from the first chamber into the stomach, waiting for the evacuation of the wax coated balloon membrane and its contents out of the body.

As an additional safety measure, a mechanical safeguard is designed into the invention to evacuate the balloon from the esophagus in the event of premature inflation. A string-like suture-strength connector can be attached to fill material located at a predetermined weak point or a predetermined manufactured perforation point of the balloon membrane. This perforated area will be filled with a fill material forming a “plug-like” structure to which the string is attached and placed in the wall of the balloon membrane to react both chemically and to physical pressure. In the event of a premature inflation in the esophagus, the string will be pulled, and a plug of the membrane will be removed, thereby permitting the gas to escape from the balloon, stopping the emergency condition. Afterwards, the balloon can be easily removed or swallowed into the stomach with a glass of water, or the balloon will elongate by the pulling pressure, and will be removed from the esophagus via the mouth.

In the alternative to simply swallowing the device, the inventor offers the option of placing the device in the stomach through a gastric tube, eliminating even the least possibility of premature inflation of the balloon device(s) within the esophagus.

Premature inflation of the device is prevented by the deliberate transfer of the liquid solution into the dissolvable capsule, assuring that the inflation will occur in the stomach, and well before any possibility of the device traveling through the pyloric sphincter and thence into the intestinal tract.

Bowel blockage is further averted by the wax that is melded into the membrane walls, creating an oily surface for easier passage. The wax is not acted upon by the stomach acids, and not only promotes easier elimination and travel of the deflated balloon through the alimentary tract, but also helps to prevent friction between the balloons and reduce friction between the balloon and the gastric mucosa, thus reducing the risk of gastric ulcers and gastric cramping.

### BRIEF DESCRIPTION OF THE DRAWINGS

FIG. 1 is a cross-sectional view of the balloon prior to activation of the capsule;

FIG. 2 is a cross-sectional view of the intragastric balloon after the chemical reaction, with the balloon being fully inflated;

FIG. 3 is a cross-sectional view of the intragastric balloon loaded into a capsule for swallowing; and

FIG. 4 is a schematic cross-sectional view showing the placement of multiple balloons, freely floating within the stomach.

### DESCRIPTION OF THE PREFERRED EMBODIMENTS

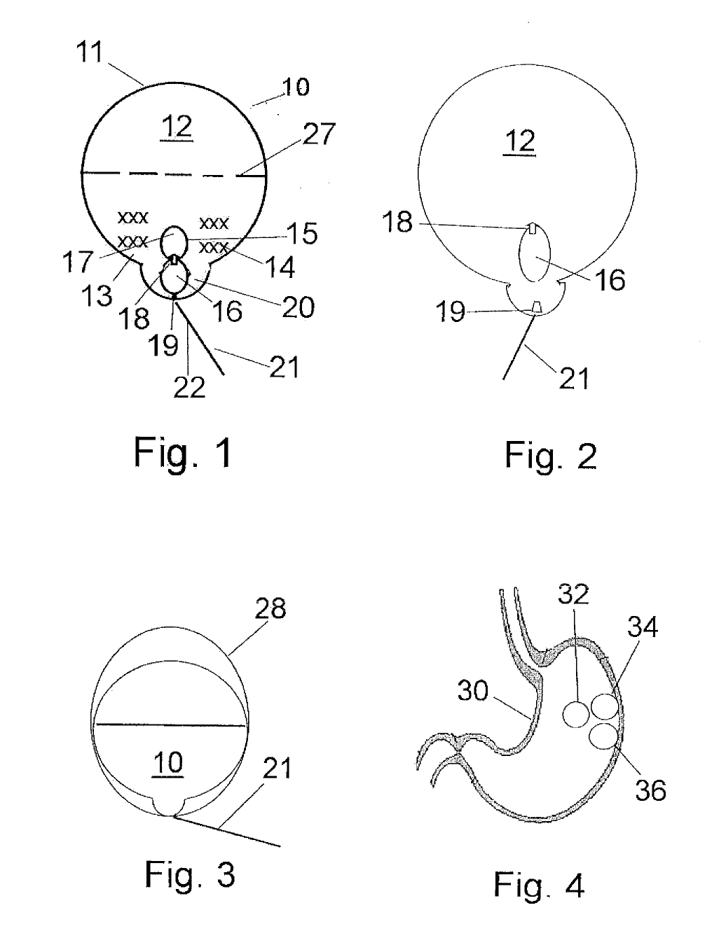

As shown in FIG. 1, a wax coated biocompatible intragastric balloon 10 is formed from a wax-coated membrane 11, and has an inner space or lumen 12, within which are housed non-toxic chemical agents, generally citric acid 13 and sodium bicarbonate 14, which react to form carbon dioxide gas to inflate the balloon. A free floating, dual chamber liquid capsule 15 is disposed in the lumen, with chambers 16 and 17, and a unidirectional valve 18 therebetween. The lumen 12 is hermetically sealed, usually with a plug or a weak point 19. A seat or pouch 20 may be created in the lumen to retain the capsule temporarily prior to the inflation of the balloon.

By squeezing the chamber 16, a liquid contained within the chamber, generally a water solution of citric acid or acetic acid, or plain water, will be transferred through the valve 18 into chamber 17, which will dissolve a predetermined period of time (e.g. 2-3 minutes) following exposure to the liquid.

A string-like suture strength connector 21 is attached to the balloon device at the engineered weak point or plug 19, that will permit reverse evacuation of the device from the esophagus and out of the mouth. As an example, this string-like connector 21 will be attached to a plug 19 in the wall of the balloon that is both chemically reactive and physically removable. In the event of successful placement and inflation of the balloon, the connector 21 will be swallowed into the stomach and dissolve quickly via chemical reaction. In the event of a premature inflation in the esophagus, however, the string is pulled, releasing the “plug” and deflating the balloon and easily retracting the device from the esophagus or swallowing the string with a glass of water.

Upon such a deflation of the device, the free floating capsule 15 will likely be evacuated from the implanted intragastric balloon membrane into the gastric cavity, to await separate elimination from the body.

The outer housing of the balloon 10 will be a soft, non-toxic wax coated capsule permitting easy ingestion of the device by swallowing. The balloon will be composed of a membrane of biocompatible polyethylene, PVC, PVCD, PET, Teflon, polypropylene, silicone or any other appropriate material, bonded with a radio-opaque substance so as to reveal the condition of the intragastric balloon at any time by the use of x-ray. The selection of the material to form the balloon, its thickness, its micro-porosity at any point of the membrane and the glue or binding substance to weld the balloon membranes together hermetically will be chosen in such a way as to permit the residency of the balloons for months within the stomach and to become deflated unilaterally subject to the degradation of either the membrane, the binding agent or other built in components of the device to permit a predetermined deflation of the device. In the alternative, an external force such as sonic waves, heat or some other directional device may be used to degrade the infrastructure of the device causing a deflation at a determined time prior to its evacuation from the stomach. Such techniques are well known in the art.

In one preferred embodiment of the invention, the suture string passes through the membrane into the lumen of the balloon. Most of the length of the string will be positioned outside the balloon and will be uncoated, and will therefore readily dissolve in the stomach acid. However, a short portion of the string 22 adjacent the balloon and incorporated into the wall thereof will be coated with a material that resists dissolution by stomach acid, for example a pectin, wax or seaweed or kelp product mixed with a binder, which coating is impervious to gastric acidity but denaturable by alkali found in the intestine. The string acts as a fuse apparatus; the outer coated portion is impermeable to the stomach acid and resists dissolution, while the inner string slowly dissolves in the stomach acid until the entire inner string dissolves into the lumen, releasing the gas from the balloon and causing deflation.

As noted, the membrane from which the balloon is formed will be coated with a wax, preferably ultra-filtered beeswax. The use of beeswax for coating a membrane is disclosed in U.S. Pat. No. 6,932,840 (incorporated herein by reference), directed to a breast implant, in which electron microscopy of a coated sac demonstrated the coating technique melds the wax into both the outer and the internal walls of the sac and permits the sac to bend in a very natural, pliable and permanent manner.

Usually, there will be a colored dye sealed within the lumen 12 to indicate the integrity of the balloon or whether one or more balloons have deflated.

The gases within the pouch will contain or be mixed with a polar substance or material that will affect a pull or attraction between the individual balloons to bond them together in a cluster, acting more like one balloon rather than independent balloons. The wax coating will insure a friction free or friction reduced environment and permit the easy escape of one deflated membrane from the cluster upon release of the plug to permit easy evacuation from the stomach.

The products, reagents, binding products and other materials used in the device will be selected from non-toxic materials known in the art of gastric balloons.

As shown in FIG. 2, upon degradation of chamber 17, the liquid passes into the lumen of the balloon, permitting mixing of the water or citric acid solution with the reagents in the lumen 12 to form the carbon dioxide or other non-toxic gas to fully inflate the intragastric balloon. Chamber 16 remains in the balloon.

The initiatory reaction creating the gas or gases to inflate the intragastric balloon within the stomach will usually be a citric acid solid and/or liquid and solid bicarbonate, preferred for their non-toxic qualities. The manual transfer of liquid within the chambers of the capsule is performed just prior to swallowing the device. The remaining chamber of the internal capsule 15 is free floating and not connected to the balloon device, permitting the free movement of this internal vessel that has no protruding or hardened components to interfere with its ultimate evacuation from the body. By being free floating, this portion of the device may be expelled from the balloon and is evacuated from the body independent from the rest of the balloon material.

The balloon remains in an inflated state until a predetermined time for degradation, at which time the balloon is emptied of gases and other contents, which are discharged into the stomach. Any remaining balloons will continue in their inflated condition until the degradation of their weak point or its fill material plug releases the gases and contents into the stomach. This process continues until one or all of the balloons are deflated and subsequently evacuated from the body.

The device will need to be “bound” for swallowing. This can be accomplished by folding the balloon device along a center-line 27 shown in FIG. 1, and loading the balloon device into, e.g. a 000 size gel capsule 28 shown in FIG. 3, or any other size capsule that is appropriate for placement via endoscope or gastroscope. It is also possible to use a wrap or other coating to hold the device together for swallowing. In either case, the coating or capsule will be reactive with the water or stomach acid and dissolve quickly once it is in the stomach. The capsule may include markings on the outer surface of pouch 20 to show where pressure is to be applied to transfer fluid between the chambers.

The safety of the capsule invention will permit multiple interventions or applications of the ingestion of the capsule/balloons within the subject individuals as frequently as is medically advisable. FIG. 4 shows multiple balloons 32, 34 and 36 disposed in stomach 30 of a person.

It is contemplated that the encapsulated balloons will be supplied to the physician in packages that contain between 1 and 5 balloons sized based upon the size of the recipient's overall height, frame structure, etc. The use of specifically sized balloons promotes patient comfort; the balloons typically come in diameters of 3.5, 4.0, 4.5 cm, up to 12 cm, usually round. All of the devices will be swallowed by the patient as explained below. The supervising physician will administer the first device by squeezing a marked end of the capsule to transfer the liquid solution from the storage chamber into the functional chamber which will dissolve in about 3-5 minutes after exposure to the liquid. Once the liquid solution is transferred, the patient immediately swallows the capsule with a sufficient amount of water to cause the capsule to enter the stomach within seconds via the normal peristalsis action of swallowing. The suture-like string is not swallowed at this moment, but is rather held outside the mouth by the medical supervising personnel until the capsule successfully enters and is inflated in the stomach. The string is swallowed when the capsule is in the stomach. Then the next capsule is swallowed and so forth until all of the capsules have been successfully administered and swallowed along with the connecting strings. Both the outer capsule or coating and the functional chambers dissolve when the balloon is in the stomach. The outer capsule is dissolved by the liquid contents of the stomach and the water being swallowed simultaneously with the administration of the capsule, and the functional chamber is dissolved by the solution which is transferred. The connector strings are dissolved by the gastric fluids.

A pectin or acid dissolving protectant will coat the string as it enters the balloon device wherein it is attached to enable emergency extraction of the balloon, and will survive at this point approximately 3-4 months. After this time, the normal breakdown of the properties of the gastric juices acting upon this part of the balloon, will cause the dissolution of this part of the coated string that is attached to the balloon, permitting the escape and the evacuation of the gases from the balloon. This will be a gradual process of erosion that will ensure a slow release of the gases from within the balloon as opposed to an “explosive,” burst of the gases from the balloon.

What is claimed is:

1. A self-inflating and self-deflating intragastric balloon device, comprising:
a biocompatible polymer membrane formed into a balloon having inner and outer surfaces, the balloon defining an interior lumen;
an internal capsule freely floating within the lumen and comprising first and second chambers with a communicating one way valve between the chambers, with the first chamber comprising a biocompatible polymer forming a gas and liquid tight housing to store a liquid solution therein, and the second chamber being composed of material capable of self dissolving or degrading in the presence of a liquid solution, wherein application of external pressure will transfer liquid from the first chamber through the valve into the second chamber;
a solid reagent disposed within the interior portion external to the internal capsule, the solid reagent reacting with the liquid solution forming a gas which inflates the balloon; and
a manufactured weakness within the membrane wall to chemically react and physically react to pressure causing a predetermined deflation.

2. The balloon device of claim 1, wherein the membrane is coated with a wax.

3. The balloon device of claim 2, wherein the wax is beeswax.

4. The balloon device of claim 1, wherein the membrane is made from a material selected from the group consisting of polyethylene, PET, Teflon, RTM, latex, polypropylene, and silicone.

5. The balloon device of claim 1, wherein the lumen comprises a seat in which the capsule is disposed.

6. The balloon device of claim 1, wherein the first chamber is made from a material selected from the group consisting polyethylene, PET, Teflon, RTM, latex, polypropylene and silicone.

7. The balloon device of claim 5, wherein the first chamber is coated with a wax.

8. The balloon device of claim 7, wherein the wax is ultra-filtered beeswax.

9. The balloon device of claim 1, wherein the second chamber is formed of gelatin.

10. The balloon device of claim 1, wherein the first and second chambers and valve are joined together by adhesive or bonding.

11. The balloon device of claim 1, wherein the balloon device is hermetically sealed with a plug constructed and arranged to provide the point of weakness for eventual deflation of the balloon device.

12. The balloon device of claim 11, wherein a string extending externally is attached to the plug, wherein pulling on the string enables removal of the plug during swallowing of the balloon device.

13. The balloon device of claim 12, wherein the string is attached to the plug by adhesive bonding.

14. The balloon device of claim 11, wherein at least a portion of the string is soluble in stomach acid.

15. The balloon of claim 14, wherein a portion of the string passing into the plug and adjacent thereto is coated to delay dissolution of the string in stomach acid.

16. The balloon of claim 15, wherein the string has a coating thereon selected from the group consisting of pectin, wax and seaweed or kelp product mixed with a binder.

17. The balloon device of claim 1, wherein the point of weakness is constructed and arranged to deflate upon exposure to stomach acid or to external physical means.

18. The balloon device of claim 1, wherein capsule contains water, citric acid, acetic acid or a mixture thereof.

19. The balloon device of claim 1, wherein at least one solid reagent is disposed in the lumen.

20. The balloon device of claim 19, wherein the solid material is at least one of sodium bicarbonate and citric acid.

21. The balloon device of claim 1, encapsulated within a capsule or coating constructed and arranged to dissolve upon swallowing.

22. The balloon device of claim 21, wherein the capsule or coating comprises a gelatin capsule.
